(19) United States
(12) Patent Application Publication
(10) Pub. No.: US 2024/0342161 A1
(43) Pub. Date: Oct. 17, 2024

(54) BISBENZYLISOQUINOLINES AS CONDITIONING AGENTS FOR IMMUNE THERAPY

(71) Applicant: Escend Pharmaceuticals, Inc., Menlo Park, CA (US)

(72) Inventors: Saira S. Bates, Napa, CA (US); Dennis Brown, Palo Alto, CA (US); Shawnya Michaels, El Granada, CA (US)

(21) Appl. No.: 18/605,652

(22) Filed: Mar. 14, 2024

Related U.S. Application Data
(60) Provisional application No. 63/453,071, filed on Mar. 18, 2023.

(51) Int. Cl. A61K 31/4745; A61K 45/06

(52) U.S. Cl. CPC A61K 31/4745; A61K 45/06

(57) ABSTRACT

The disclosed invention relates to compositions and methods of administration to a subject, with bisbenzylisoquinolines such as 6,6′,7,12-tetramethoxy-2,2′-dimethyl-berbaman (tetrandrine, TET and ES-3000), and analogs, derivatives, isomers, and modified forms such as crystalline, salt forms, or a salt of this compound with a pharmaceutically acceptable acid or in combination with other agents which modulate β-catenin and/or CaMKIIγ levels to either improve therapeutic outcome by enhancing expression of targets and/or decrease side effects of therapeutic regimens involving immunotherapies, such as checkpoint inhibitors and Chimeric antigen receptor T-cells (CAR-T).

### FIELD OF INVENTION

The disclosed invention relates to compositions and methods of administration to a subject, with bisbenzylisoquinolines such as 6,6′,7,12-tetramethoxy-2,2′-dimethyl-berbaman (tetrandrine, TET and ES-3000), and analogs, derivatives, isomers, and modified forms such as crystalline, salt forms, or a salt of this compound with a pharmaceutically acceptable acid or in combination with other agents which modulate β-catenin and/or CaMKIIγ levels to either improve therapeutic outcome by enhancing expression of targets and/or decrease side effects of therapeutic regimens involving immunotherapies, such as checkpoint inhibitors and Chimeric antigen receptor T cells (CAR-T).

### SUMMARY

The present invention provides a method sensitizing a subject to immune therapy comprising administering to the subject tetrandrine at dose of between 0.5-6 mg/kg followed by administering the immune therapy to the subject, wherein the immune therapy is administered at least 5 days after the tetrandrine is administered to the subject. Tetrandrine a bisbenzylisoquinoline such as ES-3000 (bisbenzylisoquinolines such as 6,6′,7,12-tetramethoxy-2,2′-dimethyl-berbaman (TET)), and includes analogs, derivatives, isomers, and modified forms such as crystalline, salt forms, or a salt of this compound. The subject may have, or may be at risk of having a cancer with cancer stem cell (CSC) enriched populations or a myeloproliferative disorder such as myelodysplastic syndrome (MDS), myeloproliferative neoplasms (MPN), chronic myeloid leukemia (CML), atypical chronic myeloid leukemia (aCML), or acute myeloid leukemia (AML).

### BACKGROUND OF THE DISCLOSED INVENTION

Harnessing the immune system offers great potential as a therapeutic approach to treat cancer and other diseases or therapeutic interventions (e.g. autoimmune diseases and bone marrow transplants). Immune therapeutic approaches include vaccines, adoptive T-cell therapies (TIL, LAK, NK), CAR-T and immune checkpoint inhibitors. Immune checkpoint inhibitors (anti-PD-1, anti-PD-L1, and anti-CTLA-4) are revolutionizing treatment options and expectations for patients with cancer. However, only a subset of patients respond to these treatments. In looking for differences between patients who respond compared to those that do not, researchers have found that tumors shield themselves from T cells by producing high levels of β-catenin. A comparison of patients with melanoma who did not have T cell invasion to those that did have T cell invasion showed that 49% of the tumors that blocked T cell infiltration had high levels of β-catenin.

Tumor cells without β-catenin produce the chemokine, CCL4, which attracts CD103+ dendritic cells. But CCL4 expression is suppressed by high levels of β-catenin. Active β-catenin signaling within tumor cells suppresses the recruitment of CD103+ dendritic cells by decreasing the level of CCL4. When challenged with checkpoint inhibitors, mice with melanoma that lacked β-catenin responded to treatment while mice with melanoma tumors with β-catenin did not.

Agents which reduce β-catenin expression in cancer cells have been identified. These agents include antibodies and fusion proteins which bind to proteins in the Wnt/β-catenin pathway that result in inhibition of that pathway such as OTSA101 (Centre Leon Berard, OncoTherapy Science), OMP-54F28 (OncomedPharma), Vantictumab (Oncomed Pharma), Foxy-5 (WntResearch AB) and small molecules such as tankyrase inhibitors including XAV939 and G007-LK, porcupine inhibitors including LGK974, and modulators of the TCF/LEF transcriptional subunit such as PRI-724 and ICG-001. Other small molecules which have been shown to inhibit or reduce β-catenin expression in cancer cells include niclosamide, clofazimine and salinomycin. Agents which reduce β-catenin expression in cancer cells also include agents that inhibit activated CaMKIIγ (pCaMKIIγ) or inhibit the activation of CaMKIIγ, such as bis-benzylisoquinoline alkaloids including but not limited to tetrandrine. These agents have never before been used as conditioning agents to improve responses to immunotherapies such as immune checkpoint inhibitors.

Tetrandrine (6,6′,7,12-tetramethoxy-2,2′-dimethylberbam, TET) or ES-3000 is a bis-benzylisoquinoline alkaloid, extracted from roots of the plant Stephania tetrandra S. Moore. ES-3000 (Tetrandrine) is being developed by Escend Pharmaceuticals, Inc. (Escend) for the treatment of relapsed or refractory AML and MDS. ASTX727 is an oral fixed dose combination of Decitabine (DEC) and Cedazuridine, recently FDA approved, for use in Myelodysplasia. The combination of two oral agents, with an established safety profile and potential combinatorial efficacy, would be a novel and much needed approach to treatment of intermediate and high risk MDS.

The combination of ES-3000 at varying dosages can be administered with ASTX727. The ES-3000 schedule for administration can be at one of once, twice or three times/day and can vary from about 1-14 days to about 1-7 days in combination with administration of the standard does for ASTX727 (Inqovi) for about 1-5 days, about 1-4 days, about 1-3 days and about 1-2 days. The dosage of ES-3000 can be between 20-100 mg, 25-90 mg, 30-80 mg, 40-60 mg, as well as doses from about 20 mg, 30 mg, 40 mg, 50 mg, 60 mg, 70 mg, 80 mg, 90 mg, and 100 mg as well as 105 mg, 110 mg, 115 mg, 120 mg at once, twice or three times/day. ASTX727 is the tablet referred to and is the investigational drug name for Iqovi [Decitabine (35 mg) and cedazuridine (100 mg)] tablet.

### BRIEF DESCRIPTION OF THE DRAWINGS

FIG. 1 illustrates the chemical structure of ES-3000, also known as Tetrandrine (TET).

FIG. 2 graphically illustrates the effect of TET treatment on K562 cells as percent cell viability.

FIG. 3 illustrates a PAGE showing β-catenin protein exists as a doublet in all K562 cell extracts, including untreated cells.

FIG. 4 illustrates a PAGE showing a clear beta-Actin band evident in all extracts of K562 cells treated with 0 to 40 uM tetrandrine.

FIG. 5 presents a plot depicting beta-catenin level was significantly less in extracts of K562 cells treated with >10 uM tetrandrine.

FIG. 6 presents a plot depicting beta-Actin level in extracts of K562 cells treated with up to 40 uM tetrandrine was not affected significantly by drug treatment

FIG. 7A illustrates a bar graph of samples of circulating leukemic blood cells from recipient mice following treatment with Tetrandrine or Imatinib+Tetrandrine.

FIG. 7B illustrates a bar graph of samples of leukemic stem cells from recipient mice after treatment with Tetrandrine or Imatinib+Tetrandrine on day 8 after bone marrow transplantation.

FIG. 8 graphically illustrates electrophysiology measurements of CHO cells stably transfected with hERG to examine the potential of a test article to inhibit hERG. Post-compound currents expressed as a percentage of pre-compound currents plotted against concentration for each compound.

### DETAILED DESCRIPTION

Tetrandrine (NSC 77037, d-tetrandrine) or TET is a bis-benzylisoquinoline alkaloid, extracted from roots of the plant Stephania Tetrandra S. Moore. ES-3000 (Tetrandrine derivatives thereof) is being developed by Escend Pharmaceuticals, Inc. for the treatment of relapsed or refractory (R/R) acute myeloid leukemia (AML).

Anticancer activity has been demonstrated in vitro with TET against human breast, colon carcinoma, and hepatoma cell lines with an IC₅₀ range from 1.53-10.41 uM and inhibit drug efflux and increase intracellular drug accumulation in MDR cells. TET has also been shown to inhibit wnt/β-catenin signaling and tumor growth in human colorectal (HCT116) cells. In the same study, TET exhibited synergistic anticancer activity with 5-FU and reduced migration and invasion capabilities of HCT116 cells. (He 2011, Ng 2006). In the NCI 60 cell line screen, a 50% growth inhibition (Log GI₅₀) ranged from −5.0 to −6.6 (0.25-10 UM was demonstrated in leukemia cell lines (Developmental Therapeutics Program). TET has been reported to have potent and specific activity in the reversal of P-glycoprotein mediated drug resistance. In a colorectal (CT-26) mouse xenografts, TET reduced tumor growth by inducing apoptosis (Wu 2010).

The are several known mechanism of action of TET including inhibition of calcium channels and activated big potassium channels (Wang 1995), which are commonly overexpressed in malignancies and have been demonstrated to play a role in cancer and leukemia (Arcangeli 2010, Arcangeli 2012, Wang 1995, Huber 2013 Lang 2014, Döhner 2015). TET has also been reported to reversal of P-glycoprotein (P-gp) mediated drug resistance (Zhu 2005) and though, TET itself is not a substrate of P-gp, it directly inhibits the function of P-gp in a dose-dependent manner, without altering the protein expression level of Pgp (Susa 2010). More recently, TET has been demonstrated to inhibit the Wnt/β-catenin pathway (He 2011, Xu 2012), which has been identified as a new target for AML. Wnt/β-catenin is essential for the survival and self-renewal of leukemic stem cells in acute myeloid leukemia (Simon 2005, Griffiths 2010, Wang 2010). In addition, TET has been demonstrated to bind competitively to calmodulin (CaM) and may be a novel CaM antagonist (Ma 2013). Calmodulin protein dependent kinase (CaMKIIγ) is overexpressed in leukemic stem cells and blast cells, and regulates the Wnt/β-catenin and STAT3 pathways (Si 2008).

Published clinical data support the safety and efficacy of TET in oncology patients provides another compelling rationale for clinically assessing TET in leukemia. A phase I dose escalation study was conducted by the National Cancer Institute (NCI) under IND 9613 (also known at 089613). Thirty-two patients with advanced solid tumors were administered IV doses of TET ranging from 50 to 875 mg/m2. The recommended dose for a Phase II study was 300 mg/m2 IV, single dose repeated weekly; or 200 mg/m2 IV, daily×5, repeated every 2 weeks (IND 9613 Tetrandrine Investigators Reports). Further supporting published reports include clinical studies using TET under the trademarked name CBT-1® in the US (Oldham 1998, Oldham 2000, Kelly 2012) and two studies in China (Xu 2006, Liu 2012). The drug known as CBT-1® is the same drug substance (NSC 77037) used in US Phase I study (IND #089613). CBT-1 had been publicly disclosed as NSC-77037 (tetrandrine) b (Fanelli 2016).

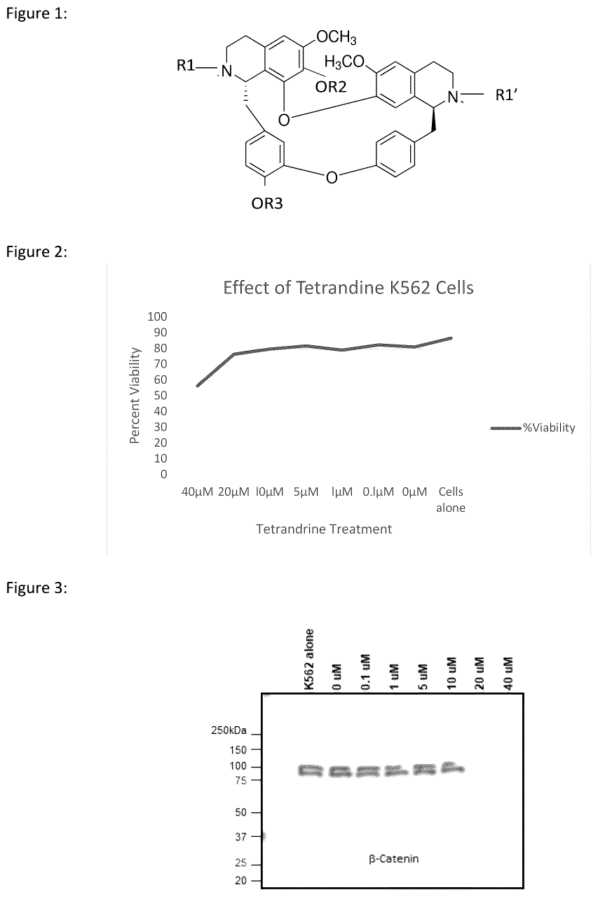

Tetrandrine is the generic name for NSC 77037, d-tetrandrine with the chemical structure written as, 6′,7,12-tetramethoxy-2,2′-dimethyl-berbaman. Chemical structure is illustrated in FIG. 1. The IUPAC name is 9,20,21,25-tetramethoxy-15,30-dimethyl-7,23-dioxa-15,30-diazaheptacyclo[22.6.2.2³,⁶.1⁸,¹².1¹⁴,¹⁸.0²⁷,³¹.0²²,³³]hexatriaconta-3,5,8(34),9,11,18,20,22(33),24(32),25,27(31),35-dodecaene, C₃₈H₄₂N₂O₆ is the chemical formula with a molecular weight of 622.76 and has CAS Registry No. 518-34-3.

The disclosed invention relates to methods and compositions to condition, pre-condition or sensitize a subject for treatment with immunotherapies such as checkpoint inhibitor therapeutics, CAR-T by administering to the subject compositions which reduce the expression of β-catenin and CaMKIIγ. Additionally, the disclosed invention relates to methods and compositions to potentiate responses to immunotherapies in a subject by administering to the subject compositions which inhibit the expression of β-catenin and/or CaMKIIγ prior to or during treatment with immunotherapies.

CaMKIIγ is a critical regulator of myeloid cell proliferation and Ca+ signaling is an important component of signal transduction pathways regulating B & T lymphocyte proliferation. CamKIIγ is invariably present in myeloid leukemia patient samples. In additional, many antigens of interest have low expression on undifferentiated or minimally differentiated AML subtypes compared to more differentiated ones. In each patient, fractions of leukemia cell exist presumably cell-renewable leukemic stem cells and exhibit low level of antigen expression. Inhibition by on Wnt/β-catenin pathway and CaMKIIγ may lead to differentiation of the relatively undifferentiated leukemic stem cells and thus enhance the expression level of myeloid lineage antigen, i.e. CAR-T targets. If so, combination treatment with ES-3000 would enhance the efficacy of the CAR-T therapies.

### Examples

Examples of immune checkpoint inhibitors for which conditioning with compositions which reduce β-catenin and/or CaMKIIγ can improve efficacy include but are not limited to antibodies that target PD-1, PD-L1 and CTLA-4.

Examples of immune checkpoint inhibitors for which conditioning with compositions which reduce β-catenin and/or CaMKIIγ can improve efficacy include but are not limited to antibodies that target PD-1, PD-L1, CTLA-4, CD20 and CD120.

Examples of compositions which inhibit β-catenin include antibodies, fusion proteins and proteins including but not limited to Wnt antibodies, OTSA101, OMP-54F28, Vantictumab, Foxy-5, FZD7 ectodomain, DKK3, sFRP4, soluble FZD8 CRD, V3Nter, and small molecules including but not limited to NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5,-14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1®, valproic acid, curcumin, resveratrol, DIF, quercetin, silymarin, carnosol, cardamonin, Decitabine, Cedazuridine, Decitabine and cedazuridine aka Astex727 (Inqovi) and salinomycin.

Examples of compositions which improve therapeutic outcome or reduce side effects of immune checkpoint inhibitors include two or more of the agents which can inhibit β-catenin including antibodies, fusion proteins and proteins including but not limited to Wnt antibodies, OTSA101, OMP-54F28, Vantictumab, Foxy-5, FZD7 ectodomain, DKK3, sFRP4, soluble FZD8 CRD, V3Nter, and small molecules including but not limited to NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5, -14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1, valproic acid, curcumin, resveratrol, DIF, quercetin, silymarin, carnosol, cardamonin, Decitabine, Cedazuridine, Decitabine and cedazuridine aka Astex727 (Inqovi) and salinomycin.

Examples of compositions which improve therapeutic outcome or reduce side effects of immune checkpoint inhibitors include vaccines, adoptive T-cell therapies (TIL, LAK, NK), CAR-T in combination with one or more agents which can inhibit β-catenin including antibodies, fusion proteins and proteins including but not limited to Wnt antibodies, OTSA101, OMP-54F28, Vantictumab, Foxy-5, FZD7 ectodomain, DKK3, sFRP4, soluble FZD8 CRD, V3Nter, and small molecules including but not limited to NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5, -14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1, valproic acid, curcumin, resveratrol, DIF, quercetin, silymarin, carnosol, cardamonin, Decitabine, Cedazuridine, Decitabine and cedazuridine aka Astex727 (Inqovi) and salinomycin.

Examples of bis-benzylisoquinoline alkaloids which can inhibit β-catenin expression in tumor cells include but are not limited to bis-benzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid may be prepared either synthetically, semi-synthetically, biochemically or through extraction processes from plant material containing the agent, including but not limited to, Stephania tetrandra and abuta.

Examples of bis-benzylisoquinoline alkaloids which can inhibit the activation of CaMKIIγ in tumor cells include but are not limited to bis-benzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid may be prepared either synthetically, semi-synthetically, biochemically or through extraction processes from plant material containing the agent, including but not limited to, Stephania tetrandra and abuta.

Pharmaceutically acceptable salts may include salts with inorganic acids, such as hydrochloride, sulphate, nitrate and phosphate, or organic acids, such as acetate, propionate, succinate, oxalate, benzoate, fumarate, maleate, methane sulphonate, isethionate, theophyllineacetate, salicylate, phenolphthalinate and methylene-bis-b-hydroxynaphthoate, or substitution derivatives of these derivatives. These may also include citrate and malate salt forms.

The disclosed invention also relates to pharmaceutical compositions consisting of bis-benzylisoquinolines such as 6,6′,7,12-tetramethoxy-2,2′-dimethyl-berbaman (tetrandrine) and derivatives, isomers and analogs, modified forms such as crystalline, salt forms, or a salt of this compound with a pharmaceutically acceptable acid, in the pure state or in the form of a composition in which it is combined with any other pharmaceutically compatible product, which can be inert or physiologically active. The medicaments according to the disclosed invention may be employed orally, parenterally, rectally, topically or as a patch.

As solid compositions for oral administration, tablets, pills, powders (gelatin capsules, wafer capsules), medical food or granules may be used. In these compositions, the active principle according to the disclosed invention is mixed with one or more inert diluents such as starch, cellulose, sucrose, lactose or silica, under a stream of argon. These compositions may also comprise substances other than diluents, for example one or more lubricants such as magnesium stearate or talc, a coloring, a coating or a varnish.

As liquid compositions for oral administration, pharmaceutically acceptable solutions, suspensions, emulsions, syrups or elixirs, containing inert diluents such as water, ethanol, glycerol, vegetable oils or liquid paraffin, may be used. These compositions can comprise substances other than diluents, for example wetting, sweetening, thickening, flavoring or stabilizing-products.

The sterile compositions for parenteral administration can preferably be solutions, aqueous or non-aqueous, suspensions or emulsions. As a solvent or vehicle, water, propylene glycol, a polyethylene glycol, vegetable oils, especially olive oil, injectable organic esters, for example ethyl oleate, or other suitable organic solvents may be employed. These compositions can also contain adjuvants, especially wetting, isotonising, emulsifying, dispersing and stabilizing agents.

The sterilization may be carried out in several ways, for example by aseptic filtration, by incorporating sterilizing agents in the composition, by irradiation or by heating. They may also be prepared in the form of sterile solid compositions which can be dissolved at the time of use in co-solvents, sterile water or any other sterile injectable medium.

The compositions for rectal administration are suppositories or rectal capsules which contain, apart from the active product, excipients such as cocoa butter, semi-synthetic glycerides or polyethylene glycols.

The compositions for topical administration can be, for example, creams, lotions, mouthwashes, nasal drops, aerosols or patches.

The doses depend on the effect sought, on the duration of the treatment and on the administration route used; they are generally between 5 and 1000 mg per day administered orally for an adult, with single doses ranging from 5 to 1000 mg of active substance. Generally speaking, the doctor will determine the appropriate dosage in accordance with the age and weight and all other factors distinctive to the subject to be treated.

Examples of methods to administer compositions which inhibit β-catenin include administration of β-catenin inhibiting compositions for at least 24 hours and up to 12 weeks prior to administration of immune therapies such as immune checkpoint inhibitors and concomitant administration of β-catenin inhibiting compositions with immune therapies such as immune checkpoint inhibitors.

### Experimental Evidence of ES-3000's Efficacy in Human Leukemias

Experiment I: Effects of Tetrandrine of β-catenin in Human Leukemia (K562) Cells

TET's ability in an in vitro assay to reduce β-catenin expression was tested in human erythroleukemic (K562) cells. The first portion of the study consisted of cell exposure to Tetrandrine and preparation of cell lysates. Testing was conducted by Applied Immunology, Menlo Park, CA.

Briefly, the method for the first portion of the study was as follows: K562 cells were grown and maintained in medium (RPMI, 10% FBS, Pen Strep and glutamate). Cells were then exposed to TET, 0, 0.1, 1, 5, 10, 20 & 40 UM for 24 hours at 37° C. Following drug exposure, cell viability was checked using trypan blue, followed by cell lysis. Supernatants were collected and test for protein concentration using a Pierce BCA kit. The protein assay tubes were read on a Spectramax at 562 nm and Softmax® software was used for protein concentration analysis. Using 8×10⁶ K562 cells per drug concentration, the protein concentration in the cell lysate was determined to be about 1 mg/ml by BCA protein assay as seen in Table 1.

TABLE 1
Effect of Tetrandrine on protein concentration in K562 Cells

| | |
|---|---|
| Treatment | Protein Recovered |

| | | |
|---|---|---|
| 40 | μM | 0.9 mg |
| 20 | μM | 1.0 mg |
| 10 | μM | 1.1 mg |
| 5 | μM | 1.0 mg |
| 1 | μM | 1.1 mg |
| 0.1 | μM | 1.1 mg |
| 0 | μM | 1.2 mg |

| | |
|---|---|
| Cells alone | 1.2 mg |

### Experiment II Effects of Tetrandrine on Beta-Catenin and Beta-Actin Levels in Human Myelogenous Leukemia K562 Cell Line

The second portion was to quantitate the levels of β-catenin protein by western blot. TET's ability in an in vitro assay to reduce β-catenin expression was tested in human erythroleukemic (K562) cells. Testing was conducted by Alamo Laboratories, San Antonia, TX.

Briefly, the method to quantitate β-catenin protein by western blot was as follows: Extracts of K562 cells (untreated and treated with various concentrations of tetrandrine) were concentrated to 0.4 ml using Centricon-3 column to yield a protein concentration of approximately 2 mg/ml. Samples were mixed with 5× Laemmli buffer and loaded on 8-16% Tris-Glycine gradient gel, then polyacrylamide gel electrophoresis (PAGE) was run at 85 volts for 100 minutes at 6° C. and proteins were transferred on PVDF membrane at 70 Volts for 85 minutes at 8° C. Membrane was blocked with 5% milk in Tris-buffered Saline containing 0.1% TW-20 (TBST). Membrane was incubated with primary antibody for 18 hours at 4° C. followed by 3 washes, 5 minutes each, with TBST and incubated with secondary antibody in TBST for 2 hours at 25° C. The membrane was washed again with TBST as described above and incubated with ECL-2 substrate for 5 minutes and scanned on Kodak Image Station. After scan, blot was stripped of primary and secondary antibodies and re-probed with anti-beta Actin antibody as described above. Protein bands in WB images were quantitated using Molecular Imaging software pack MIS-4.0 software and tabulated in Excel spreadsheet.

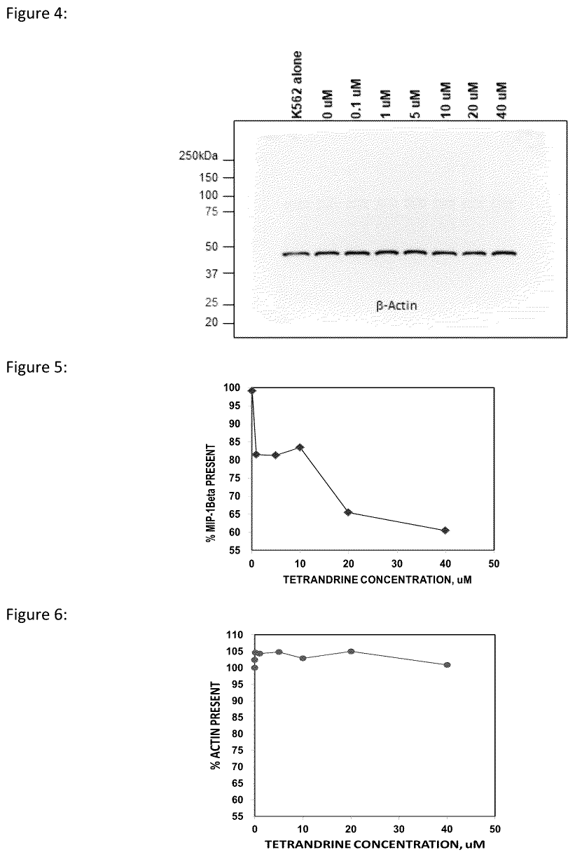

Results indicated that Tetrandrine lowers the β-catenin level in K562 cells in a dose dependent manner as seen in Table 2. It is also important to note the β-catenin protein exists as a doublet in all K562 cell extracts, including untreated cells. The doublet could arise from proteolysis, posttranslational modification or mRNA splicing. A clear β-catenin band was evident in extracts of K562 cells treated with 0 to 10 uM tetrandrine (FIG. 3). However, a convincing β-catenin band was absent in K562 cells treated with 20 and 40 uM tetrandrine. Also, a clear beta-Actin band was evident in all extracts of K562 cells treated with 0 to 40 uM tetrandrine (FIG. 4). Quantitation of the protein-bands in FIGS. 1 and 2, revealed that beta-catenin level was significantly less in extracts of K562 cells treated with >10 uM tetrandrine (FIG. 5); whereas the beta-Actin level in extracts of K562 cells treated with up to 40 uM tetrandrine was not affected significantly by drug treatment (FIG. 6). We conclude that Tet reduces β-catenin expression in K562 cells.

The effect of TET treatment on K562 percent cell viability is depicted in Table 2 and is graphically illustrated in FIG. 2.

TABLE 2
Effect of TET on K562 cell viability

| | |
|---|---|
| Treatment | % Viability |

| | | |
|---|---|---|
| 40 | μM | 56.4 |
| 20 | μM | 76.5 |
| 10 | μM | 79.7 |
| 5 | μM | 81.8 |
| 1 | μM | 79.2 |
| 0.1 | μM | 82.4 |
| 0 | μM | 81.1 |

| | |
|---|---|
| Cells alone | 86.7 |

### Experiment III: Effects of Tetrandrine in Leukemia Patient Derived Cells (In Vitro)

Study Title: In Vitro Evaluation of One Test Compound on Human Chronic
Myeloid Leukemia using Colony Forming Cell Assays
Study Report: END 01
Testing Entity: Reach Bio, Seattle, WA

An in vitro investigation was performed to determine whether TET has efficacy on human chronic myeloid leukemia (CML) stem-like cells. A colony forming cell (CFC) assay was performed on bone marrow cells from a de-novo CML patient.

Human bone marrow light density cells derived from CML patient bone marrow (Conversant Bio, Alabama), were stored in the gaseous phase of liquid nitrogen until required. On the day of the experiment, the cells were thawed rapidly, the contents of the vial were diluted in 10 mL of Iscove's Modified Dulbecco's Medium containing 10% fetal bovine serum (IMDM+10% FBS) and washed by centrifugation (approximately 1200 r.p.m. for 10 minutes, room temperature). The supernatant was discarded and the cell pellets resuspended in a known volume of IMDM+10% FBS. A cell count (3% glacial acetic acid) and viability assessment (trypan blue exclusion test) were treated with Tetrandrine (10 μM) for 14 days. After treatment, primary and secondary colonies were grown and analyzed by qPCR to determine BCR-ABL or ABL only cells.

Human erythroid (CFU-E and BFU-E) and myeloid (CFU-GM) colony enumeration were performed by trained personnel. In addition, the distribution of colony types as well as general colony and cellular morphology were analyzed. The variance in colony number detected in replicate cultures was representative of the historical coefficient of variation for colony enumeration using these types of assays. The number and distribution of colonies detected in the solvent control (0.2% DMSO) was not statistically different from the standard control (containing neither Tetrandrine or Imatinib compounds nor DMSO). For statistical analysis, colony numbers in compound-treated cultures were compared to the solvent control cultures. Potential effects of Tetrandrine and CML progenitors are presented in Table 3.

TABLE 3
Effect of ES-3000 (Tetrandrine) and Imatinib
on CML in a Methylcellulose-based Medium

| | | | Total | | CFU- | |
|---|---|---|---|---|---|---|
| | CFU-E | BFU-E | Erythroid | CFU-GM | GEMM | Total CFC |
| Standard | 10 ± 3 | 23 ± 2 | 33 ± 4 | 60 ± 4 | ND | 93 ± 0 |
| Solvent Control | 10 ± 1 | 23 ± 4 | 33 ± 4 | 56 ± 5 | ND | 89 ± 7 |
| ES-3000 30 μM | ND**** | ND** | ND** | ND** | ND | ND*** |
| ES-3000 10 μM | ND**** | ND** | ND** | 26 ± 2* | ND | 26 ± 2*** |
| ES-3000 3 μM | 9 ± 2 | 14 ± 3 | 23 ± 2 | 52 ± 5 | ND | 75 ± 6 |
| ES-3000 1 μM | 10 ± 2 | 22 ± 4 | 33 ± 3 | 54 ± 2 | ND | 86 ± 4 |
| ES-3000 0.1 μM | 11 ± 1 | 22 ± 4 | 33 ± 3 | 55 ± 5 | ND | 88 ± 8 |
| Imatinib 30 μM | ND**** | ND** | ND** | 5 ± 1** | ND | 5 ± 1*** |
| Imatinib 10 μM | ND**** | ND** | ND** | 12 ± 2** | ND | 12 ± 2*** |
| Imatinib 3 μM | ND**** | ND** | ND** | 27 ± 6 | ND | 27 ± 6** |
| Imatinib 1 μM | ND**** | ND** | ND** | 39 ± 6 | ND | 39 ± 6** |
| Imatinib 0.1 μM | 9 ± 1 | 18 ± 4 | 28 ± 5 | 57 ± 3 | ND | 85 ± 7 |

*p < 0.01;
**p < 0.001;
***p < 0.0001;
****p < 0.00001;
ND none detected

Of interest, the media formulation used for the culture of the progenitors, did not contain erythropoietin so the erythroid colonies which were detected in these cultures represent Epo independent clonal growth. ES-3000 had significant effect on CFU-GM colonies at 30 and 10 μM, while also having the same effect towards the erythroid lineage. The experimental positive control, Imatinib, also had a significant effect on CFU-GM progenitors at 30 and 10 μM but was more toxic to the erythroid lineage (30 to 1 μM) when compared to ES-3000. IC₅₀ values were determined and based on these, Imatinib was more toxic than ES-3000, Table 4.

TABLE 4
Effects of ES-3000 (Tetrandrine) and Imatinib
on Human Progenitors CML marrow

| | Erythroid IC50 ± | Myeloid IC50 ± |
|---|---|---|
| Compound | 95% CI (μM) | 95% CI (μM) |
| ES-3000 | 3.12 ± 0.01 | 9.56 ± 1.25 |
| Imatinib | 0.12 ± 0 | 2.66 ± 0.86 |

### Experiment IV: Replating Assay

This CML sample supported secondary clonal growth as determined through the replating assay (Table 3). In the solvent control cultures, 66.7% wells supported secondary growth with one or more colony and an average of three CFC per well. In the cultures initiated with colonies derived from the original cultures with Imatinib @ 3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

Experiment V: qPCR Assay for bcr-abl

To determine whether a colony was normal (bcr-abl negative and abl positive) or diseased (bcr-abl and abl positive), a TaqMan®-based qPCR test was used. In order to score the colonies positive or negative, the following scoring strategy was performed. All colonies were assayed in duplicate for each target. If a singleton reaction yielded a Ct then the reaction scored a 1. If no Ct was recorded, a score of 0 was given. From here, we used a logic test to score the colonies into four different categories: bcr-abl and abl, abl only, bcr-abl only or both negative. In order to be placed in a category, a colony must score a 1 in both reactions. However, if a colony only scored a 1 for one out of the two reactions, this colony was removed from the analysis. If a colony scored a 1 for each reaction (both bcr-abl and abl) the colony was placed into the bcr-abl and abl category, signifying it as a diseased colony. Alternatively, if a colony scored a 1 in only both of the bcr-abl reactions or a 1 in only both the abl reactions, the colony was placed in the bcr-abl only or abl only category, respectively. If the colony scored a 0 for both targets (bcr-abl and abl) then the colony was placed in the both negative category.

In total 48 primary colonies and 31 secondary replated colonies were assessed for bcr-abl and abl using qPCR. Using the scoring logic described above, three colonies were removed from the analysis. Thus, this yielded 20 out of 21 colonies (95.2%) scoring positive for both bcr-abl and abl. Seven colonies treated with Tetrandrine at 10 μM were removed from the analysis leaving 11 out of 17 colonies bcr-abl and abl positive (64.7%), One out of 17 was abl positive only (5.8%), while one out of 17 was bcr-abl positive only (5.8) and 4 out of 17 colonies were negative for both targets (23.5%). Most of the secondary colonies treated in solvent control conditions score positive for both bcr-abl and abl (10 out of 14, 71.4%) While one out of 14 (7.1%) was positive for abl only and one out of 14 (7.1%) was positive of bcr-abl only. Two out of 14 colonies scored negative for both targets. Thirteen secondary colonies treated with Tetrandrine at 10 μM were assessed. However, most colonies scored negative for both bcr-abl and abl (9 out of 10, 90%) suggesting the colony's transcript level was out of the level of detection of the assay. Only 1 out of 10 colonies scored positive for abl only (10%). No colonies scored positive for bcr-abl and abl. Further, a few secondary colonies that were treated with Imatinib at 3 μM were assessed for bcr-abl and abl. Only one out of four colonies was positive for both targets, while the remaining three colonies were negative for both targets. The results are provided in Tables 5 and 6. Table 5 indicates that in the solvent control cultures, 66.7% wells supported secondary growth with one or more colony and an average of three CFC per well. In the cultures initiated with colonies derived from the original cultures with Imatinib @3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

TABLE 5
Repeating Colony Count

| | Solvent Control | | | | Imatinib @, 3 μM | | | | ES-3000 @ 10 μM | | | |
|---|---|---|---|---|---|---|---|---|---|---|---|---|
| | 1 | 2 | 3 | 4 | 5 | 6 | 7 | 8 | 9 | 10 | 11 | 12 |
| A | 3 | 4 | 3 | 3 | 0 | 0 | 0 | 1 | 0 | 0 | 5 | 0 |
| B | 1 | 2 | 1 | 1 | 0 | 0 | 2 | 3 | 0 | 0 | 0 | 2 |
| C | 4 | 0 | 7 | 0 | 0 | 0 | 0 | 0 | 1 | 2 | 5 | 1 |
| D | 4 | 0 | 0 | 1 | 0 | 0 | 0 | 0 | 1 | 0 | 0 | 1 |
| E | 0 | 0 | 0 | 4 | 1 | 0 | 0 | 0 | 1 | 6 | 7 | 2 |
| F | 5 | 1 | 0 | 3 | 0 | 0 | 0 | 0 | 0 | 0 | 0 | 2 |

TABLE 6
bcr-abl Colony Assessment Using qPCR

| | | bcr-abl | | | | | Total |
|---|---|---|---|---|---|---|---|
| | | and abl | | bcr-abl | both | | Colonies |
| | | colonies | abl only | only | negative | Omit | Assessed |
| Primary | Solvent Control | 20 | 0 | 0 | 1 | 3 | 24 |
| | ES - 3000 @ 10 μM | 11 | 1 | 1 | 4 | 7 | 24 |
| Secondary | Solvent Control | 10 | 1 | 1 | 2 | 0 | 14 |
| | ES - 3000 @, 10 μM | 0 | 1 | 0 | 9 | 3 | 13 |
| | Imatinib @ 3 μM | 1 | 0 | 0 | 3 | 0 | 4 |

In conclusion, a semi-solid culture system which supports the proliferation of chronic myeloid progenitors, test compound ES-3000 was assessed and compared with Imatinib on primary and secondary clonal growth. This patient sample supported Epo independent growth (i.e. erythroid colonies were detected in the cultures though Epo was not added to the medium). ES-3000 and Imatinib both inhibited erythroid and myeloid colonies, though the IC50 values for Imatinib were lower than those for ES-3000. Secondary cultures were assessed by plucking individual colonies from the solvent control cultures as well as those from cultures to which Imatinib at 3 μM and ES-3000 at 10 μM were added (representing their respective IC50 values and replating them in fresh culture medium. In the solvent control cultures, 66.7% of colonies supported secondary growth as compared to 16.7% of the Imatinib treated cultures and 54.2% of the ES-3000 treated cultures. A qPCR assay was used to detect bcr-abl transcripts in plucked colonies. 95 percent of colonies from solvent control cultures were bcr-abl positive compared to 65 percent of colonies treated with ES-3000. Upon analyzing colonies from the secondary replating, most colonies treated with ES-3000 were below the limit of detection while a majority solvent control colonies remained bcr-abl positive.

### Experiment VI: Effect of Tetrandrine in a Leukemia Mouse Model

Study Title: Inhibition of CML Stem Cells with an Alkaloid that Reduces β-catenin
Study Report: In Vivo Report UMass
Testing Entity: University of Massachusetts Medical School (Dr. Li's Lab), Worchester, MA

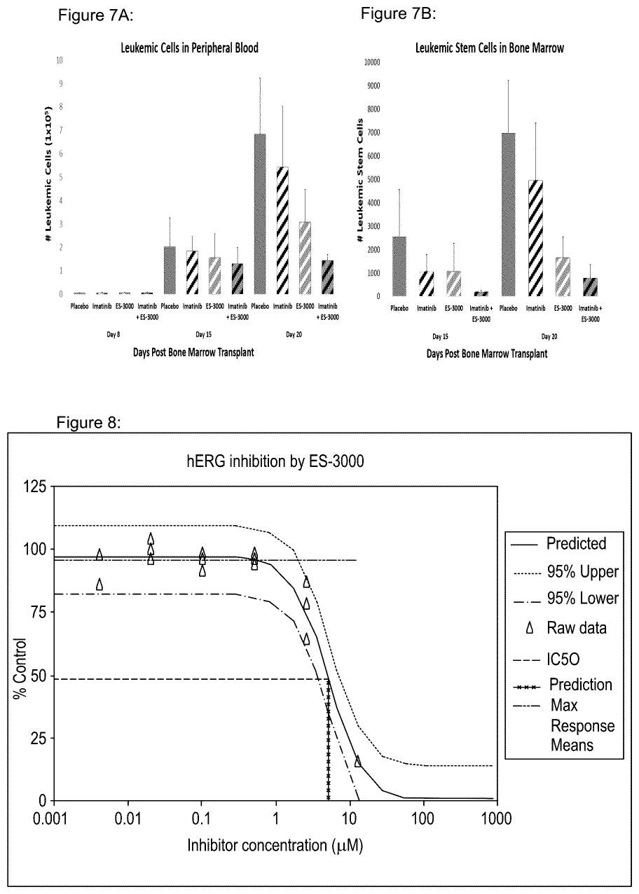

In an in vivo model, the effectiveness of Tetrandrine was tested on CML stem cells. The objective of the study was to determine the survival of leukemic cells and leukemic stem cells in response to the treatment with Tetrandrine. After priming donor C57BL/6 (B6) mice with intravenous injections of 5-fluorouracil for four days, bone marrow cells were harvested from femurs and tibia, then transfected twice with retrovirus containing MSCV-BCR-ABL-IRES-GFP. Recipient mice were lethally irradiated by two doses of 550 cGy before bone marrow transplantation by intravenous injection with 5×10⁵ cells/mouse. Blood from recipient mice was tested for disease induction one week after transduction by FACS analysis for GFP (FIG. 7A). All mice tested positive. Treatments started on day 8 after bone marrow transplantation (FIG. 7B). Mice were randomized into four groups and treated orally with vehicle [3×/day, 2×], Imatinib [100 mg/kg, 2×/day], Tetrandrine [150 mg/kg, 1×/day] or Imatinib+Tetrandrine [3×/day: 2× with Imatinib, 1× with Tetrandrine].

The results of this study demonstrated that Tetrandrine given orally once a day is superior to Imatinib given twice a day in inhibiting the development of both circulating leukemic cells and leukemic stem cells while the combination of Tetrandrine with Imatinib further improves efficacy of Tetrandrine.

### Experiment VII: Effect of Tetrandrine on CamKIIγ Activity

Study Title: ES-3000 inhibition of CAMKII gamma
Study Report: CamKII Activity Report ES-3000
Testing Entity: Institute for Rare and Neglected Diseases Drug Discovery, Mountain View, CA

An enzymatic study was conducted to evaluate the inhibition of CAMKII gamma by ES-3000 (Tetrandrine). CAMKII gamma kinase activity was measured using ATP GLO purchased from Promega following the Promega protocol. CAMKII enzyme system purchased from Promega (V3531). ADP Glo was used for detection. Assay optimized for linearity of protein and time. Conditions for inhibitor screening 25 ng CAMKII, 20 min. incubations, inhibitor or DMSO (5%) at RT in 20 uL total volume. Inhibitor, ATP (10 or 50 μM), substrates and buffer added to tubes and reactions started with enzyme. Staurosporin was used as positive control. Reactions terminated at 20 min. by adding ADP Glo reaction following manufactures instructions. All reactions were done in duplicate. Calculations of percent (%) inhibition determined by dividing the mean activity at each condition by the mean of the DMSO control times 100.

Results of individual experiments with 10 μM or 50 μM ATP are presented in Table 7 and Table 8, respectively.

TABLE 7
CamKIIγ Activity Inhibition by Tetrandrine with 10 uM ATP

| | | | | | % |
|---|---|---|---|---|---|
| Conditions | units | units | mean | % DMSO | inhibition |
| DMSO control | 4.56 | 4.15 | 4.355 | 100% | 0.1 |
| berbamine 250 uM | 2.38 | 2.3 | 2.34 | 54% | 46.3 |
| berbamine 100 uM | 2.29 | 2.32 | 2.305 | 53% | 47.1 |
| berbamine 30 uM | 2.55 | 2.967 | 2.7585 | 63% | 36.7 |
| ES-3000 250 uM | 0.34 | 0.48 | 0.41 | 9% | 90.6 |
| ES-3000 100 uM | 2.5 | 3.24 | 2.87 | 66% | 34.2 |
| ES-3000 30 uM | 2.75 | 3.43 | 3.09 | 71% | 29.1 |
| Staurosporin 10 nM | 0.93 | 0.9 | 0.915 | 21% | 79.0 |
| Staurosporin 100 nM | 0.25 | 0.15 | 0.2 | 5% | 95.4 |
| DMSO control | 4.16 | | 4.16 | 95% | 4.6 |
| ADP std - 10 uM | 0.52 | 0.44 | 0.48 | | |
| ATP std - 10 uM | 20.2 | 22.8 | 21.5 | | |

TABLE 8
CamKIIγ Activity Inhibition by Tetrandrine with 50 uM ATP

| | | | | % | % |
|---|---|---|---|---|---|
| Condition | units | units | mean | DMSO | inhibition |
| ES-3000 100 uM | 8.81 | 7.1 | 7.955 | 64% | 36 |
| ES-3000 30 uM | 10.4 | 10.7 | 10.55 | 85% | 15 |
| Staurosporin 10 nM | 9.93 | 4.64 | 7.285 | 59% | 41 |
| Staurosporin 100 nM | 0.67 | 0.6 | 0.635 | 5% | 95 |
| DMSO control | 11.5 | 13.3 | 12.4 | 1 | |
| ATP std - 10 uM | 0.34 | 0.37 | 0.355 | | |
| ADP std - 10 uM | 9.09 | 7.15 | 8.12 | | |

The results from the study showed a good signal to noise. Staurosporin was effective as a positive control and ES-3000 inhibited CAMKII gamma kinase activity in a dose dependent manner.

TABLE 9
CamKIIγ Activity Inhibition by Tetrandrine

| | Percent inhibition | Percent inhibition |
|---|---|---|
| | 10 uM ATP | 50 uM ATP |
| ES-3000 250 uM | 91 | |
| ES-3000 100 uM | 34 | 36 |
| ES-3000 30 uM | 29 | 15 |
| staurosporin 100 nM | 95 | 95 |
| staurosporin 10 nM | 79 | 41 |
| berbamine 250 uM | 46 | |
| berbamine 100 uM | 47 | |
| berbamine 30 uM | 37 | |

Experiment VIII Effects of TET on hERG
Study Title: hERG Inhibition Potential by the Test Article ES-3000
Study Report: CYP 1328_R1
Testing Entity: Cyprotex US, LLC, Watertown, MA

The purpose of this study was to evaluate the potential of a test article to inhibit hERG. The experiments are performed on an IonWorks™ HT instrument (Molecular Devices Corporation), which automatically performs electrophysiology measurements in 48 single cells simultaneously in a specialized 384-well plate (PatchPlate). All cell suspensions, buffers and test compound solutions are at room temperature during the experiment. The cells used are Chinese hamster ovary (CHO) cells stably transfected with hERG (cell-line obtained from Cytomyx, UK). A single-cell suspension is prepared in extracellular solution (Dulbecco's phosphate buffered saline with calcium and magnesium pH 7-7.2) and aliquots added automatically to each well of a PatchPlate™. The cells are then positioned over a small hole at the bottom of each well by applying a vacuum beneath the plate to form an electrical seal. The vacuum is applied through a single compartment common to all wells which is filled with intracellular solution (buffered to pH 7.2 with HEPES). The resistance of each seal is measured via a common ground-electrode in the intracellular compartment and individual electrodes placed into each of the upper wells. Electrical access to the cell is then achieved by circulating a perforating agent, amphotericin, underneath the PatchPlate and then measuring the pre-compound hERG current. An electrode is positioned in the extracellular compartment and a holding potential of −80 mV applied for 15 sec. The hERG channels are then activated by applying a depolarizing step to +40 mV for 5 sec and then clamped at −50 mV for 4 sec to elicit the hERG tail current, before returning to −80 mV for 0.3 sec. Test compound is then added automatically to the upper wells of the PatchPlate™ from a 96-well microtitre plate containing a range of concentrations of each compound. Solutions are prepared by diluting DMSO solutions of the test compound into extracellular (final DMSO concentration 0.25%). The test compound is left in contact with the cells for 300 sec before recording currents using the same voltage-step protocol as in the pre-compound scan. Quinidine, an established hERG inhibitor, is included as a positive control and buffer containing 0.25% DMSO is included as a negative control. The results for all compounds on the plate are rejected and the experiment repeated if the IC50 value for quinidine or the negative control results are outside quality-control limits. Each concentration is tested in 4 replicate wells on the PatchPlate™. However, only cells with a seal resistance greater than 50 mOhm and a pre-compound current of at least 0.1 nA are used to evaluate hERG blockade. Post-compound currents are then expressed as a percentage of pre-compound currents and plotted against concentration for each compound (FIG. 8). Testing was conducted by Cyprotex US, LLC, Watertown, MA. Experimental conditions (Table 10) and results (Table 11) are presented below.

TABLE 10
hERG Inhibition Experimental Conditions

| | | Reference | |
|---|---|---|---|
| Test Article | Test Conc. | Compound | Analytical Method |
| ES-3000 | .004-12.5 μM | Quinidine | electrophysiology |

TABLE 11
Results of hERG Inhibition by ES-3000

| | Test Conc. | | | |
|---|---|---|---|---|
| Test Article | (μM) | IC₅₀ (μM) | SE IC₅₀ | Comment |
| Quinidine | 0.01-10 | 1.66 | 0.353 | Positive Control |
| ES-3000 | 0.004-12.5 | 5.15 | 0.699 | |

This disclosed invention relates to novel compositions and methods to improve the utility of chemical agents, biological therapies including bisbenzylisoquinolines such as 6,6′,7,12-tetramethoxy-2,2′-dimethyl-berbaman (tetrandrine) and derivatives with suboptimal performance for patients with cancer, infection and immunological diseases. The disclosed invention describes the novel development of improved pharmaceutical ingredients, dosage forms, excipients, solvents, diluents, drug delivery systems, preservatives, more accurate drug administrations, improved dose determination and schedules, toxicity monitoring and amelioration, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches, the use of drug delivery systems, novel prodrugs, polymer conjugates, novel routes of administration, other agents to potentiate the activity of the compounds or inhibit the repair of suboptimal cellular effects or sub-lethal damage or to “push” the cell into more destructive cellular phases such as apoptosis. In some cases, the inventive examples include the use of these sub-optimal therapeutics in conjunction with radiations or other conventional chemotherapeutic agents or biotherapeutic agents such as antibodies, vaccines, cytokines, lymphokines, gene and antisense therapies, etc.

As used herein, “suboptimal” refers to “less that desired” and the terms “suboptimal therapy” and “suboptimal therapeutics” includes agents where Phase I toxicity precluded further human clinical evaluation, agents from Phase II trials where limited or no significant tumor responses were identified, and Phase III clinical trials, whose outcome was either medically or statistically not significant to warrant submission or approval by regulatory agencies for commercialization or commercialized agents whose response rates as a monotherapy are less than 25% or whose side-effects are severe enough to limit wider utility. In addition, it includes, allergic reaction, adverse or fatal side effects, organ damage, due to suboptimal therapeutics. Agents with suboptimal activity include but are not limited to the following: bisbenzylisoquinolines including 6,6′,7,12-tetramethoxy-2,2′-dimethyl-berbaman (tetrandrine) and derivatives, Avastin® (bevacizumab), Rituxan® (rituximab), Neavar® (sorafenib), dasatinib, imatinib, nilotinib, Provenge® (sipulcucel-T), Tarceva® (erlotinib), and Iressa® (gefitinib). More specifically, the inventive methods and compositions also focus on improvements for bisbenzylisoquinolines including 6,6′,7,12-tetramethoxy-2,2′-dimethyl-berbaman (tetrandrine) and derivatives.

Tetrandrine or TET has been shown to have multiple pharmacological activities relating to anticancer functions. Those include inhibition of Wnt/β-catenin, reduction of expression of MDR1 and inhibition of P-gp function, activation of apoptosis, and suppression of proliferation via various activation and survival signaling pathways. We have further investigated some of the pharmacologic properties most relevant to the use of TET for leukemia. Results from our studies demonstrated that TET reduced β-catenin in human (K562) leukemia cells, reduced bcr-able positive primary colonies and eliminated bcr-able positive secondary colonies in a colony forming cell assay. In a murine model of leukemia, TET reduced the development of leukemic cells in the peripheral blood and leukemic stem cells in the bone marrow of mice. In an vitro enzymatic assay, TET inhibited the activation of CaMKIIγ in a dose dependent manner.

In vitro evaluation of TET's ability to inhibit hERG, revealed an IC₅₀ of 5.2 μM, which is 13-fold higher safety for cardiac conduction-related toxicity utilizing Cmax from previous human clinical experience (Oldham 1998, Oldham 2000). Though, QT changes may be observed at concentrations lower than IC₅₀, a reasonable interpretation is that we might expect to see QT prolongation in subjects at plasma levels above 249 ng/mL, as Tetrandrine is highly protein bound.

Additionally, the Wnt/β-catenin pathway has been identified as a new target for AML and ES-3000 has shown to reduce β-catenin. More recent studies have shown that TET is able to bind competitively to calmodulin (CaM) and may be a novel CaM antagonist (Ma 2013). Calmodulin protein dependent kinase (CaMKIIγ) is overexpressed in leukemic stem cells and blast cells, and regulates the Wnt/β catenin and STAT3 pathways [Si 2008]. An evaluation of β-catenin and CaMKIIγ levels in patients to determine a correlation between response and expression levels, may provide valuable information in selecting patients who would benefit most with ES-3000 treatment.

The multiplicity of TET's mechanisms and safety profile provides an opportunity for its development as a therapeutic for relapsed or refractory AML.

This application is a U.S. Utility Patent application which claims the benefit of priority to U.S. Provisional application No. 63/453,071, filed Mar. 18, 2023, entitled “Conditioning Agents for Immune Therapy”, which is hereby incorporated herein by reference in its entirety.

### REFERENCES

Arcangeli A, Becchetti A, New trends in cancer therapy: targeting ion channels and transporters. Pharmaceuticals, 2010. 3:1202-1224.
Arcangeli A, Pillozzi S, Becchetti A, Targeting ion channels in leukemias: a new challenge for treatment. Curr Med Chem, 2012. 19 (5): 683-96.
Döhner H, Weisdorf D J, Bloomfield C D, Acute Myeloid Leukemia. The New England Journal of Medicine, 2015. 373 (12): 1136-1152.
Fanelli M, Hattinger C M, Vella S, Tayanti E, Michelacci F, Gudeman B, Barnett D, Picci P, Serra M, Targeting ABCB1 and ABCC1 with their specific inhibitor CBT-1 can overcome drug resistance in osteosarcoma. 2016, Curr Canc Drug Targets16: 261-274.
Gralla E J, Coleman G L, Jonas A M, Toxicology studies with d-Tetrandrine (NSC-77037), a plant alkaloid with vascular and lymphotoxic effects in dogs and monkeys. Cancer Chemotherapy Report Part 3, 1974. 5:79-84.
Griffiths E A, Gore S D, Hooker C M, et al., Acute myeloid leukemia is characterized by Wnt pathway inhibitor promoter hypermethylation. Leuk & Lymph, 2010. 51 (9): 1711-1719.
He B C, Gao J-L, Zhang B-Q, et al. Tetrandrine inhibits Wnt/β-catenin signalling and suppresses tumor growth of human colorectal cancer. Mol Pharmacol, 2011. 79 (2): 211-9.
Huang Y-T, Hong C-Y, Tetrandrine. Cardiovascular Drug Reviews, 1998. 16 (1): 1-15.
Huber S M, Oncochannels. Cell Calcium, 2013. 53 (4): 241-55.
Jin J, Wang F P, Wei H and Liu G. Reversal of multidrug resistance of cancer through inhibition of P-glycoprotein by 5-bromoTetrandrine. Cancer Chemotherapy and Pharmacology. 2005. 55:179-188.
Ju A, Kang Y, et al. Pharmacokinetics, tissue distribution and excretion study of tetrandrine in rats. Journal of Chinese Pharmaceutical Sciences. 2015; 24 (8).
Kelly R J, Robey R W, Chen C C, Draper D, Luchenko V, Barnett D, Oldham R K, Caluag Z, Frye A R, Steinberg S M, Fojo T, Bates S E, A pharmacodynamic study of the P-glycoprotein antagonist CBT-1 in combination with paclitaxel in solid tumors. 2012, The Oncologist. 17:512.
Lang F, Stoumaras C, Ion channels in cancer: future perspectives and clinical potential. Phil Trans R Soc B, 2014. 369:20130108.
Liu W, Zhang J, Ying C, et al. Tetrandrine combined with gemcitabine and cisplatin for patients with advanced non-small cell lung cancer improve efficacy. Int J Biomed Sci, 2012. 8 (1): 28-35.
Ma L, Wang Z, Liu S, et al. Screening calmodulin-binding ligands using intensity-fading matrix-assisted laser desorption/ionization mass spectrometry. Rapid Commun Mass Spectrom, 2013. 27: p. 1527-1534.
National Cancer Institute, Annual Progress Report for IND #089613, 1976.
Ng L T, Chiang L-C, Lin Y-T, et al. Antiproliferative and apoptotic effects of Tetrandrine on different human hepatoma cell lines. Am J Chin Med, 2006. 34 (1): 125-35.
Oldham R K, Reid W K, Preisler H D, Barnett D. A phase I and pharmacokinetic study of CBT-1 as a multidrug resistance modulator in the treatment of patients with advanced cancer. Cancer Biother Radiopharm. 1998; 13:71-80.
Oldham R K, Reid W K, Barnett D, Phase I study of CBT-1 and Taxol in patients with Taxol resistant cancers. 2000, Cancer Biother Radiopharm. 15 (2): 153-159.
Si J, Collins S J, Activated Ca2+/Calmodulin-dependent protein kinase Ily is a critical regulator of myeloid leukemia cell proliferation. Cancer Res, 2008. 68 (10): 3733-3742.
Simon M, Grandage V L, Linch D C and Khwaja A. Constitutive activation of the Wnt/β-catenin signalling pathway in acute myeloid leukaemia. Oncogene 2005. 24:2410-2420.
Susa M, Choy E, Yang C, Schwab J, Mankin H, Hornicek F, Duan Z. 2010. Multidrug resistance reversal agent, NSC77037, identified with a cell-based screening assay. J Biomol Screen. 15, 287-296.
Wang G, Lemos J R, Tetrandrine: a new ligand to block voltage-dependent Ca2+ and Ca2+-activated K+ channels. Life Sci, 1995. 56 (5): 295-306.
Wang Y, Krivtsov A, Sinha A, The wnt/β-catenin pathway is required for the development of leukemia stem cells in AML. Science, 2010. March 26; 327 (5973): 1650-1653.
Wu J-M, Chen Y, Chen J-C, Lin T-Y, Tseng S-H, Tetrandrine induces apoptosis and growth suppression of colon cancer cells in mice. 2010, Cancer Letters. 287:187-195.
Wu K, Zhou M, Wu Q-X, Yuan S-X, Wang D-X, Jin J-L, Huang J, Yang J-Q, Sun W-J, Wan L-H, He B-C, The role of IGFBP-5 in mediating the antiproliferation effects of Tetrandrine in human colon cancer cells. 2015, Int. J Oncology. 46:1203-1213.
Xu W-L, Shen H-L, Ao Z-F, Combination of Tetrandrine as a potential-reversing agent with daunorubicin, etoposide and cytarabine for the treatment of refractory and relapsed acute myelogenous leukemia. Leuk Res, 2006. 30:407-413.
Xu X H, Gan Y C, Xu G B, Chen T, Zhou H, Tang J F, Gu Y, Xu F, Xie, Y Y, Zhao X Y, Xu R Z, Tetrandrine citrate eliminates imatinib-resistant chronic myeloid leukemia cells in vitro and in vivo by inhibiting Bcr-Abl/β-catenin axis. J Zhejiang Univ-Sci B (Biomed & Biotechnol), 2012 13 (11): 867-874.
Zheijiang Jinhua Conba Bio-Pharma, Co. Ltd., (Jinhua, China), Tetrandrine Tablet Package Insert 2014.
Zhu X, Sui M, Fan W, In vitro and in vivo characterizations of Tetrandrine on the reversal of p-glycoprotein-mediated drug resistance to paclitaxel. 2005, Anticancer Research. 25:1953-1962

What is claimed:

1. A method of sensitizing a subject to immune therapy comprising administering to the subject tetrandrine at dose of between 0.5 mg/kg-6 mg/kg followed by administering the immune therapy to the subject, wherein the immune therapy is administered at least 5 days after the tetrandrine is administered to the subject.

2. A method according to claim 1, wherein said immune therapy is selected from a group of antibodies or proteins including Wnt antibodies, OTSA101, OMP-54F28, Vantictumab, Foxy-5, FZD7 ectodomain, DKK3, sFRP4, soluble FZD8 CRD or V3Nter.

3. A method according to claim 1, wherein said immune therapy is selected from a group of small molecules including NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5, -14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine, ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1, valproic acid, curcumin, resveratrol, DIF, quercetin, silymarin, carnosol, cardamonin, Decitabine, Cedazuridine, Decitabine and cedazuridine aka Astex727 (Inqovi) or salinomycin.

4. A method according to claim 1, wherein said immune therapy comprises a combination of two or more of the agents which can inhibit β-catenin including antibodies, fusion proteins and proteins including but not limited to Wnt antibodies, OTSA101, OMP-54F28, Vantictumab, Foxy-5, FZD7 ectodomain, DKK3, sFRP4, soluble FZD8 CRD, V3Nter, and small molecules including but not limited to NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5, -14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1, valproic acid, curcumin, resveratrol, DIF, quercetin, silymarin, carnosol, cardamonin, Decitabine, Cedazuridine, Decitabine and cedazuridine aka Astex727 (Inqovi) and salinomycin.

5. A method according to claim 1, wherein said immune therapy comprises vaccines, adoptive T-cell therapies (TIL, LAK, NK) or CAR-T therapy in combination with one or more agents which can inhibit β-catenin including antibodies, fusion proteins and proteins including but not limited to Wnt antibodies, OTSA101, OMP-54F28, Vantictumab, Foxy-5, FZD7 ectodomain, DKK3, sFRP4, soluble FZD8 CRD, V3Nter, and small molecules including but not limited to NSC668036, PNU 74654, 2,4-diamino-quinazoline, FJ9, 3289-8625, IWR, IWP, IC261, 1α,25 (OH)2D (3)-Vitamin D3, glucocorticoids, iCRT3-,-5, -14, NC043, retinoids, PKF118-310, CGP049090, PKF115-584, PKF222-815, PKF118-744, ICG001, CCT036477, XAV939, Acyl hydazones, HQBA, molecules with 2,3,6-trisubstituted pyrido[2,3,-b] pyrazine core skeletons, carnosic acid, CCT031374, ICRT-3,5,14, NC043, BC2059, AV-65, niclosamide, clofazimine ibuprofen, indomethacin, doxycycline, minocycline, homoharringtonine (Synribo®), bruceantin, aspirin, ECGC, sulindac, celecoxib, bis-benzylisoquinoline alkaloids, tetrandrine, 6,6′,7,12-tetramethoxy-2,2′-dimethyl-berbaman, CBT-1®, valproic acid, curcumin, resveratrol, DIF, quercetin, silymarin, carnosol, cardamonin, Decitabine, Cedazuridine, Decitabine and cedazuridine aka Astex727 (Inqovi) and salinomycin.

6. A method according to claim 1, wherein said composition comprises a member of the bis-benzylisoquinoline family having the following structural formula: 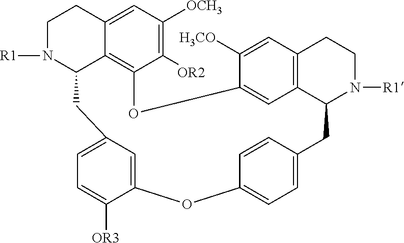
where R1 and R1′ are the same or different short chained carbon based ligand including without limitation, CH₃, CO₂CH₃ or H; and R2 is CH₃ or C₂H₅; and R3 is CH₃ or hydrogen, has the “S” isomeric configuration at the C-1′ chiral carbon location.

7. A method according to claim 1, wherein said composition is administered for at least 24 hours and up to 12 weeks prior to administration of immune therapy.

8. A method according to claim 1, wherein said composition is administered prior to administration of immune therapy.

9. A method according to claim 1, wherein said composition is administered concomitant to administration of immune therapy.
